(19) United States
(12) Patent Application Publication
(10) Pub. No.: US 2003/0021768 A1
(43) Pub. Date: Jan. 30, 2003

(54) Viral mutants that selectively replicate in targeted human cancer cells

(76) Inventors: Yuqiao Shen, Orinda, CA (US); Terry Hermiston, Corte Madera, CA (US); Ali Fattaey, San Francisco, CA (US)

Correspondence Address: Gregory Giotta, Ph.D., Vice President and Chief Legal Counsel, ONYX Pharmaceuticals, Inc., 3031 Research Drive, Richmond, CA, 94806, US

(21) Appl. No.: 10/191,922

(22) Filed: Jul. 9, 2002

Related U.S. Application Data
(60) Provisional application No. 60/307,576, filed on Jul. 23, 2001.

(51) Int. Cl. A61K 48/00; C12N 7/00; C12N 15/861

(52) U.S. Cl. 424/93.2; 435/456; 435/235.1

(57) ABSTRACT

Oncolytic viruses are described that have favorable anti-cancer activity and that are produced by random mutagenesis and subsequent bio-selection on cancer cells wherein the viruses are preferably adenoviruses having at least one mutation in the i-leader sequence of the viral major late transcriptional unit.

### FIELD OF THE INVENTION

The invention described herein relates generally to the field of molecular biology, and more specifically to adenoviral vectors that have prophylactic or therapeutic applications.

### BACKGROUND OF THE INVENTION

Conditionally replicating viruses represent a promising new class of anti-cancer agents [Ref(s): 1-5: Martuza, 2000; Alemany, 2000; Curiel, 1997; Kirn, 1996; Kirn, 2000]. Derivatives of human adenovirus type 5 (Ad5) have been developed that selectively replicate in, and kill, cancer cells. The prototype of such viruses, ONYX-015, has demonstrated encouraging results in several phase I and phase II clinical trials with patients having recurrent head-and-neck cancer and patients having liver metastatic disease [Ref(s): 6-12: Bischoff, 1996; Heise, 2000; Kirn, 2001; Kirn, 1998; McCormick, 2000; Nemunaitis, 2001; Nemunaitis, 2000].

A lesson learned from the on-going clinical trials is that efficacy rather than toxicity of the adenovirus appears to limit its therapeutic benefit [Ref(s): 7-9 and 11-14: Ganly, 2000; Heise, 2000; Kirn, 1998; Kirn, 2001; Nemunaitis, 2001; Nemunaitis, 2000; Nemunaitis, 2001]. To date, no dose-limiting toxicity has been observed.

One strategy to enhance the clinical efficacy of oncolytic virues is to combine them with other therapies, eg. standard chemotherapy [Ref(s): 7-8: Kirn, 2001; Heise, 2000], or to arm them with anti-cancer genes [Ref: 15: Hermiston, 2000], such as anti-angiogenesis factors, cytotoxic agents, pro-drug converting enzymes, or cytokines, etc. Another approach, which can be combined with chemotherapy and anti-cancer genes, is to genetically alter the virus to render it more potent, i.e. replicate faster, produce more viral progenies, and enhance tissue and/or cell type specificity etc. The goal here being to generate therapeutic viruses that kill cancer cells more rapidly, selectively, and eventually eradicate the cancer.

Two general genetic approaches have been taken for developing oncolytic viruses with enhanced anti-cancer properties. The first is targeted genetic manipulation, in which certain viral genes, or regulatory elements (i.e. promoters) are deleted, or foreign genes inserted, etc. Although this approach has been successfully utilized to construct many novel viruses (eg. [Ref(s): 16-20: Yu, 2001; Fueyo, 2000; Howe, 2000; Maxwell, 2001; Samoto, 2001]), its application is limited by the requirement of a thorough understanding of the biology of that virus. Even in the case of Ad5, one of the most extensively studied viruses, such information is not always available or complete. Thus, targeted genetic manipulations are in many cases very difficult to make. The second approach is genetic selection under carefully controlled conditions. Viruses selected in this fashion grow preferentially under that particular condition (for examples, see [Ref(s): 21-26: Beck, 1995; Berkhout, 1993; Berkhout, 1999; Domingo, 1995; Polyak, 1998; Soong, 2000]. In essence, this is a natural evolution process, only occurring under carefully controlled conditions in the laboratory.

It should be apparent that viruses offer a powerful means for treating cancer. Thus, viruses that selectively replicate in, and kill neoplastic cells would be an invaluable weapon in a physician's arsenal in the battle against cancer.

### SUMMARY OF THE INVENTION

A first object of the invention is to describe genetically altered viruses with favorable anti-cancer activity.

A second object of the invention is to describe genetically altered viruses with favorable anti-cancer activity produced using random mutagenesis and subsequent bio-selection on cancer cells wherein the mutagenesis causes at least one mutation in a viral transcriptional unit that enhances the ability of the mutated virues to replicate in and kill cancer cells.

A third object of the invention is to describe genetically altered adenoviruses with favorable anti-cancer activity produced using random mutagenesis and subsequent bio-selection on cancer cells wherein the mutagenesis causes at least one mutation in a viral transcriptional unit that enhances the ability of the mutated virues to replicate in and kill cancer cells.

A fourth object of the invention is to describe genetically altered adenoviruses, preferably Ad 5, with favorable anti-cancer activity produced using random mutagenesis and subsequent bio-selection on cancer cells wherein the mutagenesis causes at least one mutation in the i-leader sequence of the viral major late transcriptional unit.

A fifth object of the invention is a description of methods and compositions for treating cancer using mutagenized adenovirus having one or more mutations in the i-leader sequence of the viral major late transcriptional unit, and optionally, the addition of select genes to the virus that encode medically beneficial proteins. Such genes would preferrably include heterologous genes including negative selection genes, and/or genes that encode cytokines.

A sixth object of the invention is a description of altered adenoviruses, preferably Ad 5, with favorable anti-cancer activity produced using random mutagenesis and subsequent bio-selection on cancer cells wherein the mutagenesis causes at least one mutation in the i-leader sequence of the viral major late transcriptional unit, and such mutation is combined with mutations associated with other oncolytic viruses.

These and other objects of the present invention will become apparent to one of ordinary skill in the art upon reading the description of the various aspects of the invention in the following specification. The foregoing and other aspects of the present invention are explained in greater detail in the drawings, detailed description, and examples set forth below.

### BRIEF DESCRIPTION OF THE DRAWINGS

FIG. 1. (A). Wild-type Ad5 was mutagenized by treatment with NaNO₂. Infectivity of the treated virus was examined by plaque assay on 293 cells, and plotted as a function of incubation time. (B). Representative plaque assays on HT29 cell monolayer, 5 days post infection with wild-type Ad5 or bio-selection viruses. (C). Microscopic view (40×) of representative plaques formed on HT29 cells by Ad5 or ONYX-201.

FIG. 2. (A). Cytopathic effects of HT29 cells either mock infected (Mock) or infected (at a multiplicity of infection of 10) with wild-type Ad5, ONYX-201 and -203. Pictures were taken 3 days post-infection. (B). Cytolytic activity in HT29 cells was examined using MTT assays. HT29 cells were infected with serial 3-fold dilutions of various viruses, ranging from MOI of 30 to MOI of 1.5×10⁻³. MTT assays were performed 5 days after infection as described.

FIG. 3. Kinetics of HT29 cytotoxicity. HT29 cells were infected with Ad5, ONYX-201 and ONYX-203 at various MOIs. At different time points post infection, percentage of viable cells was assessed by MTT assay and plotted vs. time.

FIG. 4. HT29 cells were infected at MOIs of 10, 1, 0.1 and 0.01. At different time points after infection, cells and culture media were collected. (A). Virus yields. 4×10⁴ HT29 cells were infected. Total virus yields (cells and culture media combined) were determined by plaque assay on 293 cells. (B). Viral DNA replication. DNAs were extracted using Boold DNA Extraction Kit (Qiagen), digested with Hind III, and resolved on 0.8% agarose gels. After Southern transfer, the blots were hybridized with probes prepared using DIG High Prime DNA labeling kit (Roche Biochemicals). Ad5 genomic DNA served as template for probe synthesis. (C). Viral gene expression. Cell extracts were prepared at various days post infection (dpi) and separated by SDS-PAGE. Expression of E1A and viral late proteins (structural proteins) were examined by Western blot analysis. M, mock infected.

FIG. 5. Cytolytic activity in various tumor cells. Tumor cells were seeded in 96-well dishes at a density of 3,000 cells/well. Twenty-four hours after seeding, they were infected with Ad5, ONYX-201, ONYX-203, or the virus pool that was passaged in HT29 cells (VHT29). Infections were conducted using serial 3-fold dilutions of each virus, starting from an MOI of 30

FIG. 6. Cytolytic activity in primary normal human cells. Cells were grown in 96-well dishes and were infected with Ad5, ONYX-201 and -203 in serial 3-fold dilutions. (A). Quiescent mammary epithelial cells, MEC. (B). Quiescent small airway epithelial cells, SAEC. (C). Proliferating microvescular endothelial cells, MVEC. Similar experiments were carried out multiple times in various primary normal human cells (proliferating or non-proliferating). Similar results were obtained, only 3 representative experiments are shown here. (D). Relative cytotoxicity in “matched” tumor and normal cells. MTT assays were performed on primary MEC and a mammary cancer cell line, MB468, using Ad5, ONYX-201, 203, and ONYX-015. The IC50 value was defined as that MOI which resulted in 50% cell killing. These values were then plotted relative to that of Ad5 for each virus as follows: IC50 (Ad5)/IC50 (test virus). Therefore, the relative activity of Ad5 in normal and tumor cells is 1.

FIG. 7. (A). Recombination schemes. Various recombinant viruses were constructed as described above. The exclamation marks indicate mutations present in each recombinant virus. Restriction sites for Pme I, Bam HI, and Spe I are indicated on the viral genomes. (B). Cytolytic activity of the recombinant viruses was examined in HT29 cells using MTT assays.

### DETAILED DESCRIPTION OF THE INVENTION

All publications, including patents and patent applications, mentioned in this specification are herein incorporated by reference to the same extent as if each individual publication was specifically and individually indicated to be incorporated by reference in its entirety.

### Definitions

Unless defined otherwise, all technical and scientific terms used herein have the same meaning as commonly understood by one of ordinary skill in the art to which this invention belongs. Generally, the nomenclature used herein and the laboratory procedures described below are those well known and commonly employed in the art.

Standard techniques are used for recombinant nucleic acid methods, polynucleotide synthesis, and microbial culture and transformation (e.g., electroporation, lipofection). Generally, enzymatic reactions and purification steps are performed according to the manufacturer's specifications. The techniques and procedures are generally performed according to conventional methods in the art and various general references (see generally, Sambrook et al., Molecular Cloning: A Laboratory Manual, 2nd. edition (1989) Cold Spring Harbor Laboratory Press, Cold Spring Harbor, N.Y.) which are provided throughout this document. The nomenclature used herein and the laboratory procedures in analytical chemistry, organic synthetic chemistry, and pharmaceutical formulation and delivery, and treatment of patients.

The term “adenovirus” as referred to herein indicates over 40 adenoviral subtypes isolated from humans, and as many from other mammals and birds. See, Strauss, “Adenovirus infections in humans,” in The Adenoviruses, Ginsberg, ed., Plenum Press, New York, N.Y., pp. 451-596 (1984). The term preferably applies to two human serotypes, Ad2 and Ad5.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset with 200 bases or fewer in length. Preferably oligonucleotides are 10 to 60 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides. The term “naturally occurring nucleotides” referred to herein includes deoxyribonucleotides and ribonucleotides. The term “modified nucleotides” referred to herein includes nucleotides with modified or substituted sugar groups and the like known in the art.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). Various methods of labeling polypeptides and glycoproteins are known in the art and may be used. Examples of labels for polypeptides include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), enzymatic labels (e.g., horseradish peroxidase, b-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains epitope tags).

The term “sequence homology” referred to herein describes the proportion of base matches between two nucleic acid sequences or the proportion amino acid matches between two amino acid sequences. When sequence homology is expressed as a percentage, e.g., 50%, the percentage denotes the proportion of matches over the length of sequence that is compared to some other sequence. Gaps (in either of the two sequences) are permitted to maximize matching; gap lengths of 15 bases or less are usually used, 6 bases or less are preferred with 2 bases or less more preferred.

DNA regions are operably linked when they are functionally related to each other. For example: a promoter is operably linked to a coding sequence if it controls the transcription of the sequence; a ribosome binding site is operably linked to a coding sequence if it is positioned so as to permit translation. Generally, operably linked means contiguous and, in the case of leader sequences, contiguous and in reading frame.

The term “selectively hybridize” referred to herein means to detectably and specifically bind. Polynucleotides, oligonucleotides and fragments of the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10. The two sequences or parts thereof are more preferably homologous if their amino acids are greater than or equal to 50% identical when optimally aligned using the ALIGN program.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

Methods for the construction of adenoviral mutants are generally known in the art. See, Mittal, S. K., Virus Res., 1993, vol: 28, pages 67-90; and Hermiston, T. et al., Methods in Molecular Medicine: Adenovirus Methods and Protocols, W. S. M. Wold, ed, Humana Press, 1999. Further, the adenovirus 5 genome is registered as Genbank accession #M73260, and the virus is available from the American Type Culture Collection, Rockville, Md., U.S.A., under accession number VR-5.

Generally, adenovirus vector construction involves an initial deletion or modification of a desired region of the adenoviral genome, preferably the Ad5 genome, in a plasmid cassette using standard techniques.

The instant invention presents adenoviral mutants, preferably Ad5, that replicate significantly better than the parental virus in cancer cells wherein the mutants derive their beneficial anti-cancer activity from at least one mutation in a viral transcriptional unit which is preferably the i-leader sequence of the viral major late transcriptional.

The preferred method for producing the viruses is by random mutagensis, and subsequent passage, or selection, on cancer cells. The preferred materials and methods used to realize the instant invention are as follows:

Cells and viruses: All human cancer cell lines were obtained from American Type Culture Collection (ATCC) and were propagated as monolayer cultures in Dulbecco's modified Eagle's medium (DMEM) supplemented with 10% fetal bovine serum (FBS), 2 mM L-glutamine, 100 μg/ml non-essential amino acids (NEAA), 10 U/ml penicillin and 10 μg/ml streptomycin. Primary human normal cells were obtained from Clonetics, Inc., and were propagated under conditions recommended by the manufacturer. Wild-type adenovirus type 5 (Ad5) was obtained from ATCC and propagated in 293 cells. ONYX-201 and -203 and their derivatives were propagated in HT29 cells until the last proliferation step, in which they were grown in 293 cells. All viruses were purified by CsCl-gradient banding method, and titrated by plaque assay on 293 cells. In several cases plaque assays were performed on HT29 cells as well as on 293 cells, and the results from both were consistent. Infections of cancer cells were performed in DMEM supplemented with 2% FBS, 2 mM L-glutamine, 100 μg/ml NEAA, 10 U/ml penicillin and 10 μg/ml streptomycin. Infections of normal cells were performed in their recommended growth media.

Random mutagenesis. Random mutagenesis of Ad5 with nitrous acid was performed as previously described [Ref(s) 27-30: Fried, 1965; Williams, 1971; Praszkier, 1987; Klessig, 1977]. Briefly, wild-type Ad5 was treated with 0.7 M NaNO₂ in 1M acetate buffer, pH 4.6. The reaction was terminated at various time points by addition of 4 volumes of ice-cold 1 M Tris-Cl (pH 7.9). The virus was then dialyzed overnight against 20 mM PBS (pH 7.2)/10% glycerol, and stored in −80° C. Infectivity of the treated virus was examined by plaque assay on 293 cells.

Bio-selection. Mutagenized Ad5 was repeatedly passaged on selected human cancer cell lines representing various human cancers. In all cases, infections were carried out in T-185 tissue culture flasks containing approximately 10⁷ adherent cells in 25 ml of culture medium (2% FBS). For the first round of passaging, cells were infected at a multiplicity of infection (MOI) of 1. Tissue culture media were harvested at the very initial sign of visible cytopathic effect (CPE). In the subsequent passagings, 1 ml, 0.1 ml, or 0.01 ml of the harvested media from the previous passaging was used as inocula. Cultures that began to show CPE at 3 to 5 days post inoculation were considered effective, and media were collected at the initial sign of CPE. This strategy allowed us to avoid infection with too many virus particles, which may reduce the effectiveness of bio-selection, or too little virus, which reduced the complexity of the viral population. Passaging was carried out for 6 to 20 rounds, depending on the cell lines.

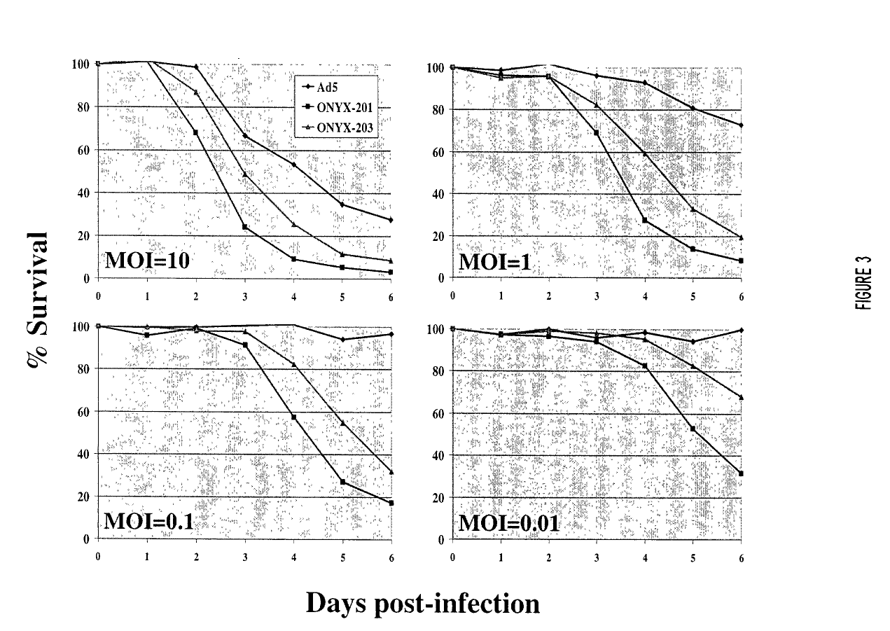

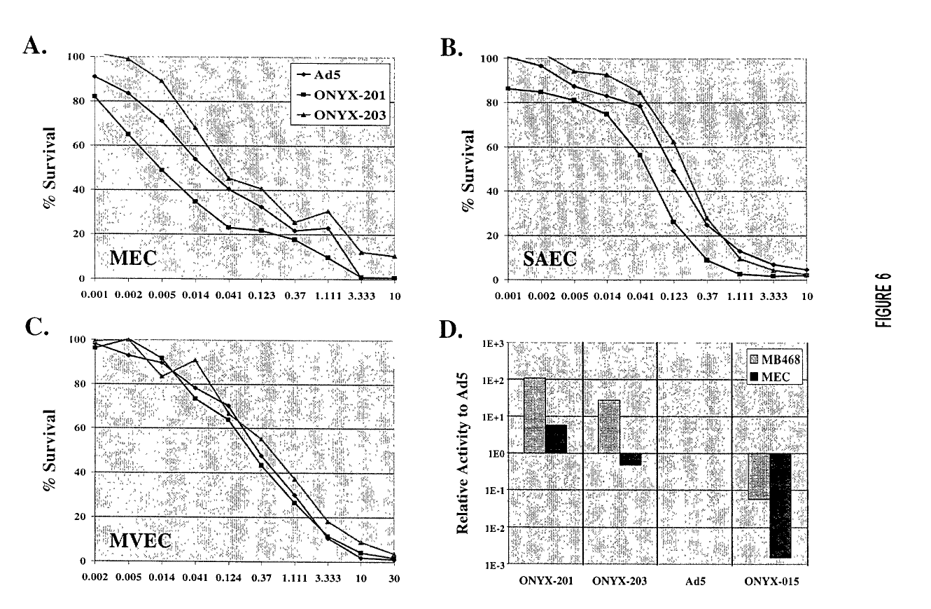

Cytolytic Assay. Viral cytolytic activities were examined using MTT assay as described [Ref: 31: Shen, 2001]. Briefly, cells were seeded into 96-well plates at a density of 3,000 cells per well in appropriate growth media. Infections were performed at 24 hours after seeding with various viruses. In most cases, infections were carried out in quadruplet with serial three-fold dilutions of the viruses. A total of 10 dilutions were used for each virus, starting at an MOI of 30 and ending at an MOI of 1.5×10⁻³. Some of the MTT assays with primary human cells (FIG. 6) had a starting MOI of 10 and an ending point at 5×10⁻⁴. Cytolytic assays described in FIG. 3 were conducted in triplet at MOIs of 10, 1, 0.1 and 0.01. Infected cells were incubated at 37° C. and colorimetric reactions were performed at indicated time points, using CellTiter 96® Non-Radioactive Cell Proliferation Assay (Promega) according to the manufacturer's instructions. Cells that were mock infected were used as negative controls, and set as reference (100% survival).

Burst Assays. HT29 cells were seeded in 24-well dishes at 4×10⁴ cells per well. After they attached to the plate, cells were infected with Ad5, ONYX-201 and -203 at indicated MOIs. After an incubation period of 90 minutes, unattached viruses were removed, and cells were washed once with phosphate buffered saline (PBS). Infected cells were then incubated at 37° C. in DMEM supplemented with 2 mM L-glutamine, 100 μg/ml NEAA, 10 U/ml penicillin, 10 μg/ml streptomycin and 2% FBS. At indicated time points after infection, cells and culture media were harvested separately. Cell fractions were frozen-thawed 3 times to release virus particles, lysates were cleared by centrifugation. Total virus yield (cell and medium combined) was determined by plaque assay on 293 cell monolayers.

Viral DNA replication. HT29 cells were infected with Ad5, ONYX-201 and -203 at various MOIs. At indicated times post-infection, cells and culture media were harvested, and DNAs were extracted from combined cell and medium fractions using Qiagen's Boold DNA Extraction Kit. DNAs were digested to completion with Hind III and digested DNAs were resolved on 0.8% agarose gels. After Southern transfer, DNA blots were hybridized with probes prepared using DIG High Prime DNA labeling kit (Roche Biochemicals). Purified Ad5 genomic DNA was used as template for probe synthesis.

Western blot analysis. HT29 cells were either mock infected or infected with Ad5, ONYX-201 and -203 at various MOIs. At indicated times post-infection, cells were harvested and lysed in SDS gel loading buffer (100 mM Tris-Cl [pH 6.8], 5 mM EDTA, 1% SDS, 5% β-mercaptoethanol). Proteins were fractionated by electrophoresis on 4-20% protein gels (Bio-Rad). After electrophoresis, the proteins were electrophoretically transferred to nylon membranes. Blots were then incubated with antibodies diluted in PBS containing 1% dry milk and 0.1% Tween-20, and visualized by ECL (Amersham). Anti-E1A antibody M73 (Calbiochem) was diluted 1:500; a polyclonal rabbit anti-Ad5 (structural proteins) antibody was used at 1:10,000.

DNA Sequencing. Genomic DNAs of ONYX-201 and -203 were purified from CsCl gradient-banded virus particles. Briefly, virus particles were lysed by incubation at 37° C. in 10 mM Tris-HCl (pH 8.0), 5 mM EDTA, 0.6% SDS and 1.5 mg per ml of pronase (Sigma). Lysed particles were extracted twice with phenol/chloroform, and DNA was precipitated with ethanol. Genome of ONYX-201 was sequenced by Lark Technologies, Inc., Texas. Genomic DNA of ONYX-203 was sequenced at Onyx Pharmaceuticals, Inc.

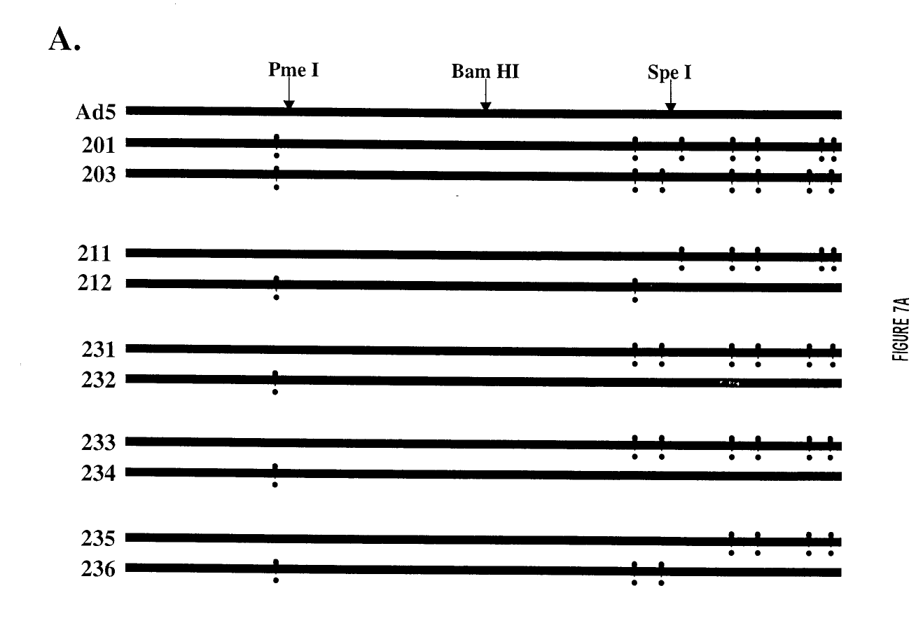

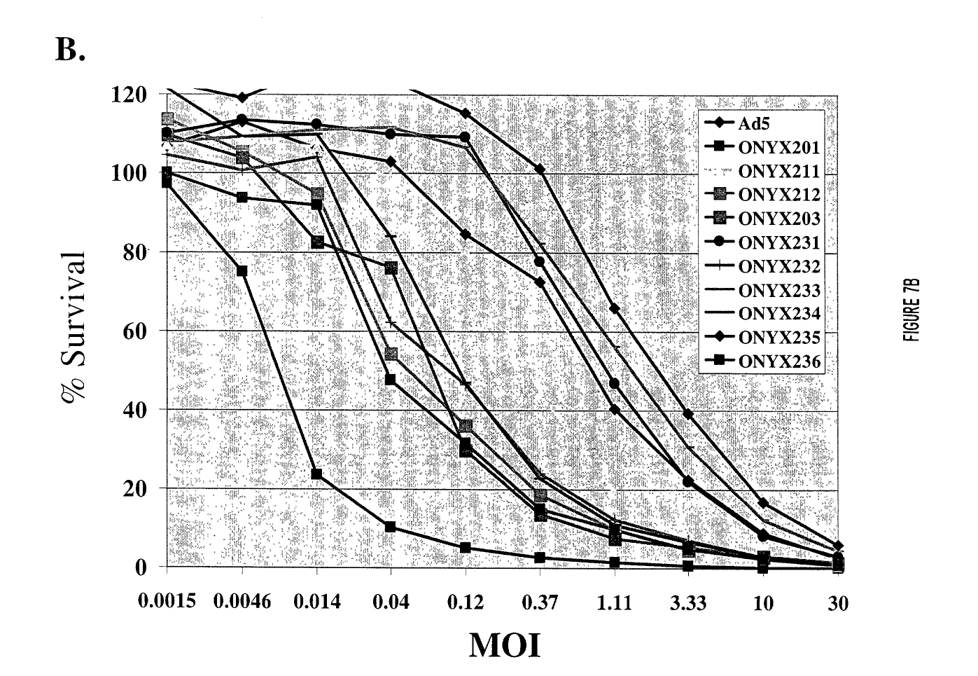

Construction of Recombinant Viruses. Genomic DNAs of Ad5, ONYX-201 and -203 were purified from CsCl gradient-banded virus particles. For construction of ONYX-211 and 212, genomic DNAs from Ad5 and ONYX-201 were both digested to completion with Spe I, which cuts only once within the viral genome. Digested DNAs were mixed in equal amount and ligated in the presence of T4 DNA ligase at room temperature overnight. This ligation mixture was then transfected into 293 cells using FuGene reagent (Promega). Plaques derived from this transfection were isolated and screened by DNA sequencing. Proper clones were purified by an additional round of plaque assay. ONYX-231 through -236 were constructed in a similar fashion, except that DNAs from Ad5 and ONYX-203 were digested with Pme I, BamH I or Spe I, respectively (see FIG. 7). All recombinant viruses were confirmed by sequencing the regions surrounding the 7 mutation sites in ONYX-201 and -203.

### Description of Preferred Embodiments

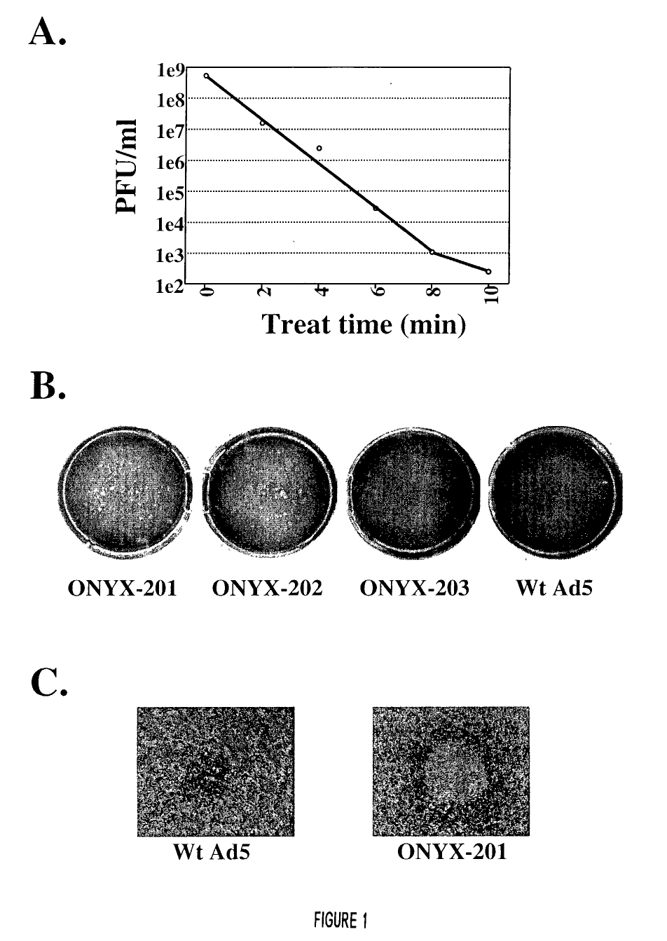

Estimation of the mutation frequency. In order to maximize the efficiency of bio-selection, wild type Ad5 was treated with nitrous acid (NaNO₂), a chemical mutagen. Treatment with NaNO₂ dramatically reduced the viability of the virus (FIG. 1A). A treatment of 6 minutes at room temperature reduced the viability of Ad5 by a factor of 2×10³ which was consistent with earlier observations. To estimate the mutation frequency in this treated virus population (6-minute treatment), we picked 22 individual plaques formed on A549 cell monolayer by this virus population, isolated viral DNAs, and amplified a 340-base pair fragment in their E1A region by PCR. The PCR products were analyzed by DNA sequencing. Out of the 22 individual virus isolates analyzed, two had a single base pair mutation. We therefore estimated that mutations occurred at a frequency of 2.7×10⁻⁴, which means that, on average, each viral genome (36 kilobases in size) has approximately 10 single point mutations. This is very close to the actual number of mutations in the two viral genomes we have analyzed (see below).

Bio-selection. Mutagenized Ad5 was independently passaged in a number of human cancer cell lines representing various human cancers. Passaging procedure is described in Materials and Methods. Importantly, tissue culture media were harvested at the very early sign of cytopathic effects (CPE), and used to inoculate the next passaging. This procedure was carried out for 6 to 20 rounds, depending on the cell lines. To test the effectiveness of this bio-selection protocol, the following experiment was conducted. Two viruses, wild-type Ad5 and LGM, a derivative of Ad5 that contains the green fluorescent protein (GFP) gene in place of the E1B-55K gene, were mixed at a ratio of 1:1. This mixture was passaged on U2OS cells using the protocol described above. It has been shown previously that adenovirus mutants defective in the E1B-55K gene grow poorly in U2OS cells. After each passaging, the culture medium was harvested, and the relative abundance of the two viruses was examined by Southern blot analysis. After 3 passages in U2OS cells, Ad5 clearly became the dominant species, constituting >95% of total virus. There was no detectable LGM in culture medium from passage 5. This experiment demonstrated the selection power of the passaging protocol we used in this study.

Characterization of the bio-selection viruses. During the course of serial passaging, we noticed the virus pool that was passaged on a human colon cancer cell line, HT29, showed progressive improvement in its cytolytic capacity on this cell line. HT29 was quite resilient to infection by Ad5, usually took more than 4 days to show significant CPE even at MOI of 10. We therefore characterized this virus population after it was passaged in HT29 for 19 passages. This virus population is referred to as “VHT29”. VHT29 was first analyzed by plaque assay on nine cell lines: HT29, 293, A549 and H2009 (lung cancer), DU145 and PC-3 (prostate), MB231 (breast), Panc-1 (pancreas), and Hlac (Head and neck). Wild-type Ad5 were used as a control. We noticed that a subset of plaques (approximately 50% of the total plaques) formed by VHT29 on HT29 cell monolayer were exceptionally big (3-5 mm in diameter after 7 days) compared to the plaques formed by Ad5 (<2 mm in diameter). Interestingly, VHT29 did not form extraordinary large plaques on any other cell lines.

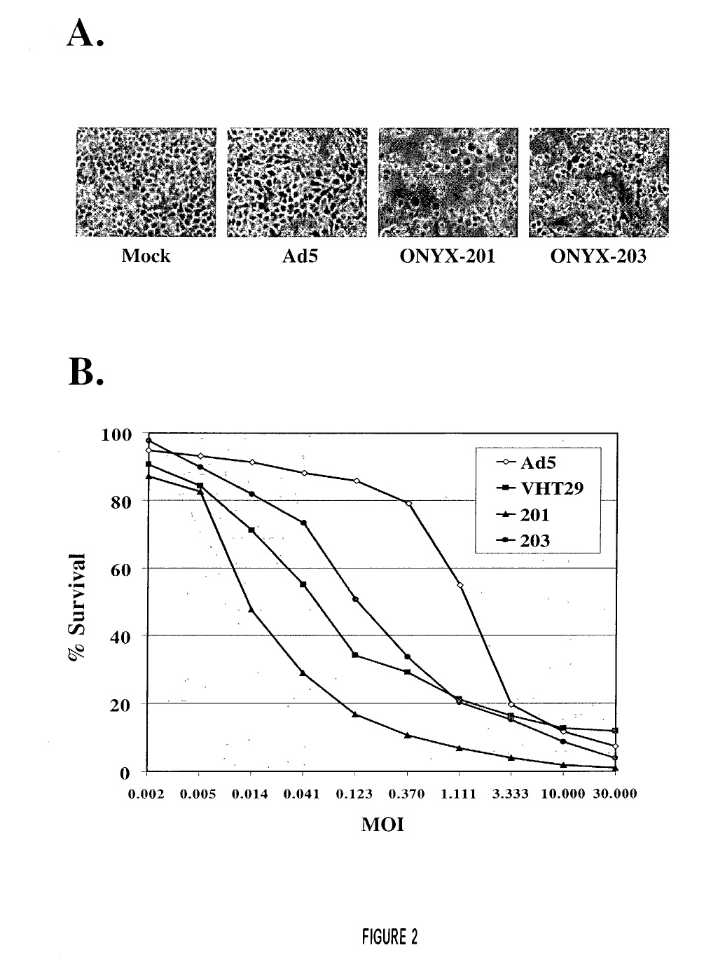

Twenty large plaques were isolated for further investigation. Viruses from these plaques were propagated in HT29 cells, and examined again by plaque assay on HT29 cells. Among others, three viral isolates (ONYX-201, -202 and -203) produced uniformly large plaques on HT29 cell monolayer when compared to Ad5 (FIG. 1B). ONYX-201 and 203 were selected for further analysis. To demonstrate the potency of these viruses, HT29 cells were infected with ONYX-201, -203 and Ad5 at an MOI of 10. Cells infected with ONYX-201 and -203 showed CPE a lot faster than cells infected with Ad5 (FIG. 2A). Between the two mutant viruses, ONYX-201 was more potent than ONYX-203 in cytolysis. In addition, we also noticed that the morphology of the cells infected with ONYX-201 and -203 was different from cells that were infected with Ad5. Ad5-infected cells tended to stick to one another, displaying a typical “grape-vine” like morphology characterized of an adenovirus infection, whereas cells infected with ONYX-201 and -203 were well separated from one another, and cells were swollen with smooth surface.

Using an MTT assay, we compared the cytolytic activity of ONYX-201, -203, Ad5. The order of cytolytic activity is ONYX-201>-203>Ad5 (FIG. 2B). This result was repeated in many independent experiments. In most cases, ONYX-201 was approximately 500- to 1000-fold more active than Ad5, whereas ONYX-203 was about 30- to 50-fold more active than Ad5. Interestingly, the cytotoxicity of VHT29, the virus pool from passaging in HT29 cells, was between those of ONYX-201 and ONYX-203 (FIG. 2B), consistent with the fact that VHT29 was a mixture of viral individuals with varying cytotoxicty.

It should be noted that this difference in cytolysis was unlikely to be due to an inaccurate assessment of virus titers. Ad5, ONYX-201, 203 and VHT29 were all titrated in HT29 cells as well as in 293 cells, and the results were consistent. In FACS analysis and immunofluorescent staining studies using cells that were infected at a very low MOI of 0.01, fraction of cells that were positive for the viral early protein E1A was similar for Ad5, ONYX-201 and -203. This also proved that the titers for these viruses were accurate. The particle to infectious unit (particle:pfu) ratios for these viruses were not significantly different, all between 10 and 50.

Kinetics of cell lysis and viral replication. Next we examined the progression of HT29 cytolysis as a function of time and MOI. At an MOI of 10, ONYX-201 lysed cells more rapidly than ONYX-203, which killed cells more efficiently than Ad5 (FIG. 3). The differences were somewhat modest at this MOI. However, as we reduced MOI, the differences became more and more drastic. At MOIs of 0.1 and 0.01, cells infected with Ad5 showed no clear signs of CPE during the course of the experiment, and cell viability only dropped slightly. However, cells infected with ONYX-201 and -203 at these low MOIs displayed unambiguous CPE between day 3 and day 4, and cell viability dropped dramatically (FIG. 3).

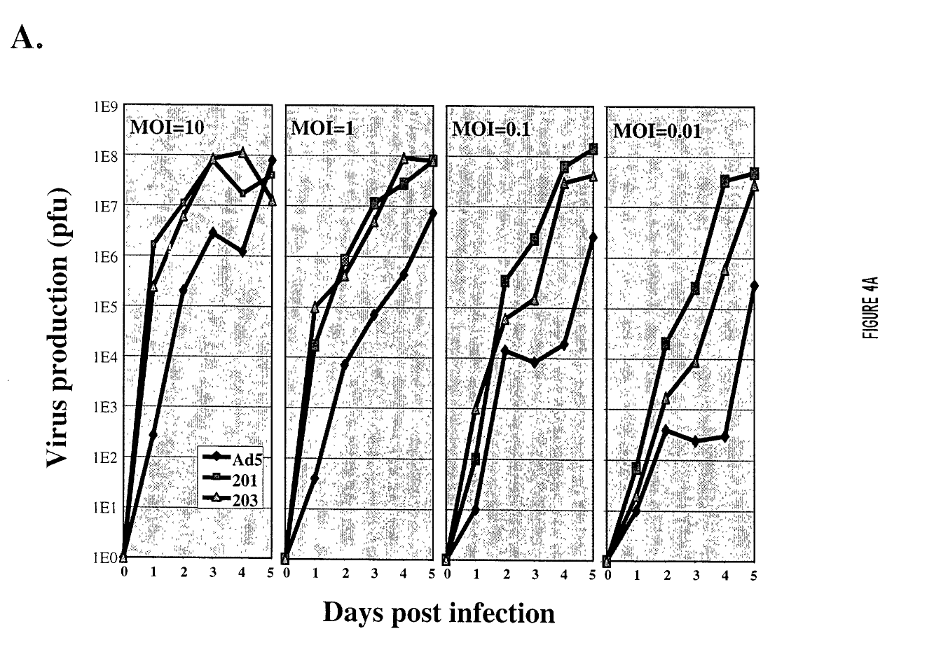

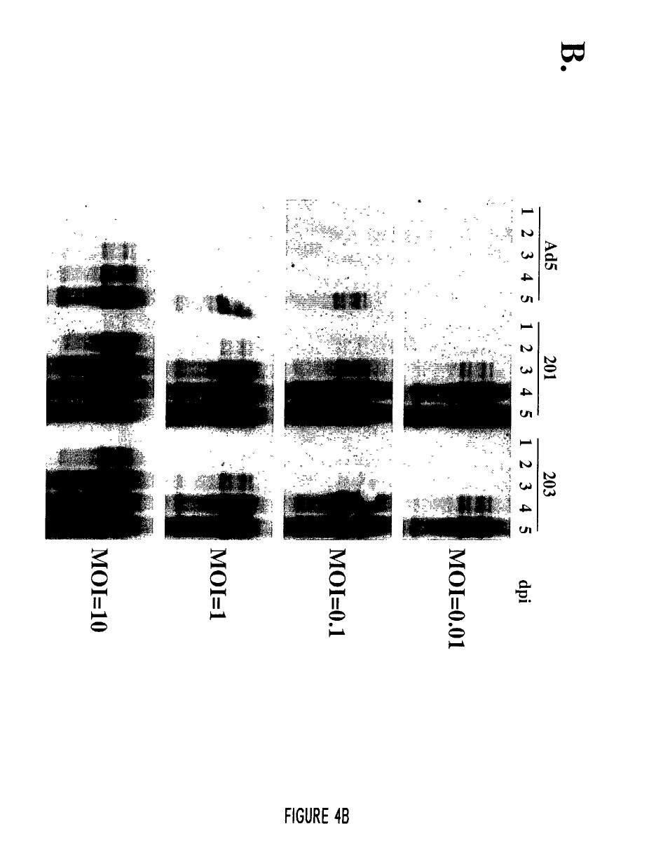

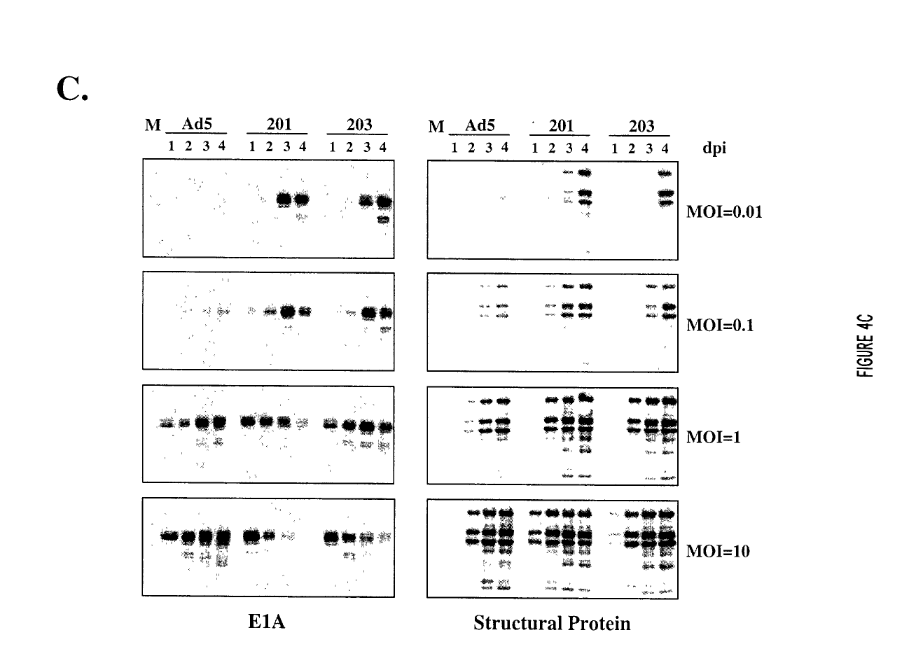

Efficient cytolysis may result from a number of possible mechanisms, eg. greater infectivity, faster rate of replication, larger progeny yield per cell, etc. To explore the mechanism, we examined the kinetics of viral progeny production, DNA replication, and early as well as late gene expression, all at various MOIs from 0.01 to 10. Immunofluorescent staining studies indicated that Ad5, ONYX-201 and -203 were not different in terms of infectivity, as E1A expression was equivalent in all cases. At an MOI of 10, in which >90% of the cells were infected, the final yields of virus progeny were similar for ONYX-201, -203 and Ad5 (FIG. 4A). However, it took Ad5 much longer time to reach the maximun production level (5 days vs. 3 days. FIG. 4A). This result suggested that virus yields per cell were similar, but the rates of production were significantly different. Indeed, viral DNA replication occurred more rapidly (FIG. 4B) and viral gene products accumulated faster (FIG. 4C) for ONYX-201 and -203 as compared to Ad5. These observations were also consistent with our hypothesis. As we lowered MOI, the difference among ONYX-201, -203 and Ad5 in progeny production, DNA replication and protein accumulation became more substantial, consistent with the cytolysis results. It is likely that at low MOIs (MOI of 1 and below), multiple rounds of infection took place, and the difference in the rate of viral replication was “amplified” through each replication cycle.

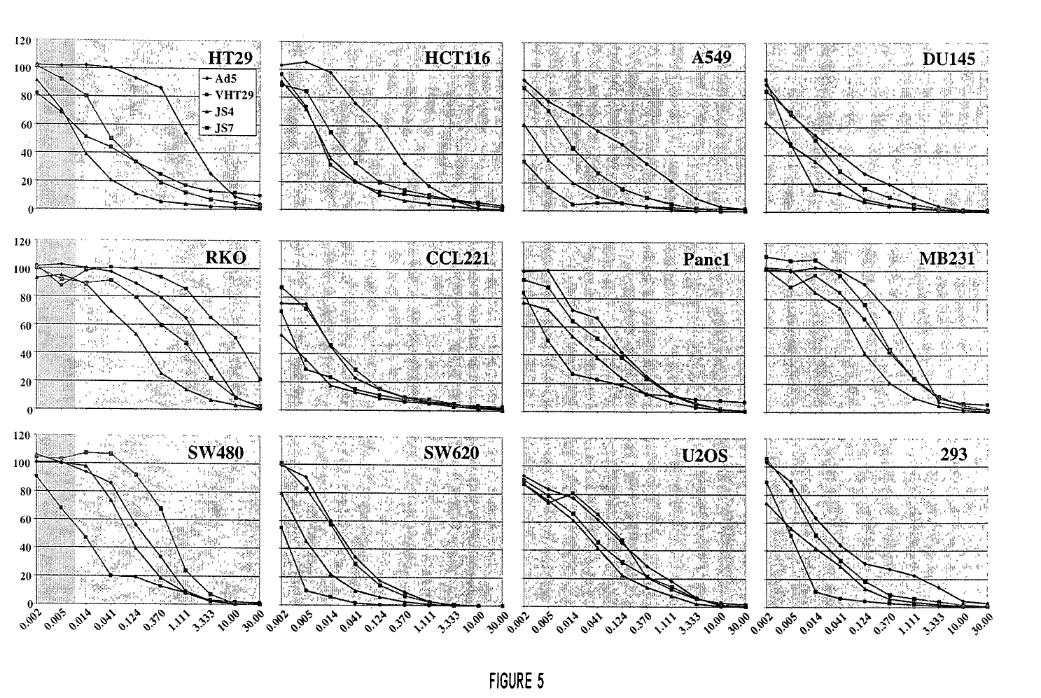

Cytotoxicity in other human cancer cells. We examined the cytotoxicity of ONYX-201 and -203 in a number of other human cancer cell lines to test whether their greater cytolytic activity was restricted only to HT29. Twelve cancer cell lines, including 6 derived from human colorectal cancers, HT29, HCT116, CCL221, RKO, SW480 and SW620, and 6 other human tumor cell lines of different origins, A549 (lung), DU145 (prostate), MB231 (breast), Panc-1 (pancreas), U2OS (osteosarcoma) and 293 (transformed human kidney cells), were tested in the MTT assays. Results from a representative MTT assay was shown in FIG. 5. ONYX-201, -203 and VHT29 displayed a significantly higher cytolytic activity than Ad5 in HT29 cells, consistent with results in FIG. 2. Significantly, these viruses showed substantially higher cytolytic activity than Ad5 in many other cancer cell lines. For example, in A549 and in HCT116 cells, ONYX-201 and 203 are significantly more potent in cell killing than Ad5, whereas in DU145 and Panc-1 cells, the difference was marginal (FIG. 5). In all cell lines tested, ONYX-201 was more active than Ad5. We conclude that the viruses that were selected on HT29 cells had accumulated mutations that allow them to specifically replicated very efficiently in HT29 cells, and in many other cancer cells as well.

Cytolytic activity in normal cells. The cytotoxicity of ONYX-201 and 203 in primary human normal cells was examined by MTT assay. In all the cell types that we have tested so far (SAEC, PrEC, MEC, REC, HuVEC and MVEC), whether they are proliferating or non-proliferating, ONYX-201 and 203 were quite equivalent to Ad5. ONYX-201 was usually slightly more active than Ad5, whereas ONYX-203 was slightly more attenuated than Ad5. Representative results from SAEC, MEC and MVEC are shown in FIG. 6. Using a pair of “matched” cells from the same tissue, mammary cancer cell line MB468 and primary normal mammary epithelial cells (MEC), we assessed the artificial “therapeutic index” of each virus. Cytolytic activity in MB468 and in MEC of each virus was normalized to that of Ad5, and plotted in FIG. 6D. Thus, the height of the bars represents the cytotoxicity as compared to Ad5; and the difference between the blue bar and the purple bar indicates therapeutic index, defined as the relative activity in tumor cells divided by that in normal cells. ONYX-201 and -203 showed same tumor to normal specificity as ONYX-015, but were substantially more potent than either ONYX-015 or Ad5. These data raise an suggest that while ONYX-201 and -203 have accumulated the ability to efficiently infect and lyse HT29 and many other tumor cells, their cytolytic activity in primary human normal cells did not significantly change. In combination, these features offered increased tumor cell selectivity of these oncolytic agents.

Mutation mapping. Moving towards an understanding of the molecular mechanisms of ONYX-201 and -203, we had their entire genomes sequenced. These mutations, along with their possible consequences, were listed in Table 1. Both ONYX-201 and -203 contain seven single point mutations, consistent with our prediction of 10 mutations per genome. Four mutations were shared by both viruses, while the rest of the mutations were unique to each virus.

To delineate which mutation(s) was responsible for the phenotypes described above, we constructed a series of recombinant viruses harboring various mutations identified in ONYX-201 and -203. An illustration of these recombinant viruses and the construction strategy is shown in FIG. 7A. The cytolytic activity of these recombinant viruses was compared by MTT assays on HT29 cells. Results from the MTT assay (FIG. 7B), combined with the morphological inspection of the infected HT29 cells, indicated that all viruses containing the mutation at nucleotide 8350 (C to T) displayed the super-killing phenotype. ONYX-212, -232, -234 and -236 all had activities similar to that of ONYX-203, including morphology of the infected cells. On the other hand, ONYX-231, -233 and -235 behaved the same as wild-type Ad5. Therefore, we conclude that the C to T mutation at nucleotide 8350 was necessary and sufficient for the increased cytolytic activity of ONYX-203. This mutation was also necessary to account for the superior cytolytic activity of ONYX-201.

Intending not to be bound by any particular theory to account for the anti-cancer activity of the invention viruses, we note that the C to T mutation at nucleotide 8350 completely accounts for the cytolytic activity of ONYX-203, and is required for the activity of ONYX-201. This mutation is located in the i-leader of the major late transcription unit of Ad5 [Ref(s): 32-36: Falvey, 1983; Symington, 1986; Virtanen, 1982; Lewis, 1983; Akusjarvi, 1981]. The i-leader sequence is spliced to a subset of L1 mRNA, which predominantly encodes the 52/55K protein, and may modulate expression of the 52/55K protein [Ref(s): 36-39: Soloway, 1990; Akusjarvi, 1981; Persson, 1981; Lucher, 1986]. The i-leader itself contains an open reading frame that codes for a 145-amino acid protein, i-leader protein [Ref(s): 32-36: Falvey, 1983; Symington, 1986; Virtanen, 1982; Lewis, 1983; Akusjarvi, 1981]. The exact roles of the 52/55K protein and the i-leader protein in adenovirus replication are not clear. They are the only proteins encoded in the major late transcription unit that are expressed prior to viral DNA replication [Ref(s): 33, 36, 39-40: Lewis, 1985; Akusjarvi, 1981; Lucher, 1986; Symington, 1986]. This suggests that these two proteins may have an important role in the initiation of viral DNA replication. The C to T mutation at nucleotide 8350 changes the codon for Gln at amino acid 125 to a stop codon UAG, thus eliminating the last 21 amino acids of the i-leader protein. It is thus thought that this changes the expression of the i-leader protein and the 52/55K protein, and thus affects viral replication to produce favorable anti-cancer activity.

It is important to note that viruses of the instant invention may be constructed on the genetic backgrounds of other oncolytic viruses to yield a virus with further enhanced anti-cancer activity. The preferred viruses would be adenoviral mutants which substantially lack the ability to bind p53 resulting from a mutation in the gene that encodes the E1B-55K protein. Such viruses generally have some, or all of the E1B-55K region deleted. U.S. Pat. No. 5,677,178, inventor, McCormick, describes, among other things, adenoviral mutants that lack a viral oncoprotein, that is E1B-55K protein or E4 orf6. Also, U.S. Pat. No. 6,080,578 describes, among other things, adenoviral mutants that have deletions in the region of the E1b55K protein that is responsible for binding p53. Another preferred oncolytic adenovirus is one that has a mutation in the E1A region is described in U.S. Pat. Nos. 5,801,029 and 5,972,706. Thus, mutations in the E1B-55K and/or E1A regions of adenovirus may be combined with the mutations of the instant invention adenoviruses, and preferably adenovirus having a mutation in the i-leader sequence as described above.

Further, the viruses of the instant invention may be imparted an enhanced degree of tissue specificity by putting the replication of the viruses under the control of a tissue specific promoter as described in U.S. Pat. No. 5,998,205. Also, the replication of the invention viruses may be put under the control of an E2F responsive element as described in U.S. patent Ser. No. 09/714,409. The latter affords a viral replication control mechanism based on the presence of E2F, and is thus distinct from the control realized by a tissue specific promoter. Both a tissue specific promoter, or an E2F responsive element are operably linked to an adenoviral gene that is essential for the replication of said adenovirus.

Therapeutic Methods

Therapy of disease, preferably cancer, may be afforded by administering to a patient a composition comprising adenoviruses of the invention, and further comprising a heterologous gene, such as a negative selection gene or other genes, for example, cytokines, to augment the cancer killing activity of the invention viruses. Examples would include cytosine deaminase, thymidine kinase, and gm-csf, respectively. Such genes may be inserted in different regions of adenovirus as is known in the art, and preferably in the E1 and/or E3 regions.

The viruses of the instant invention may be combined with chemotherapy or X-ray therapy to treat cancer. The preferred chemotherapeutic agent is cisplatin, and the preferred dose may be chosen by the practitioner based on the nature of the cancer to be treated, and other factors routinely considered in administering cisplatin. Preferably, cisplatin will be administered intravenously at a dose of 50-120 mg/m² over 3-6 hours. More preferably it is administered intravenously at a dose of 80 mg/m² over 4 hours. A second chemotherapeutic agent, which is preferably administered in combination with cisplatin is 5-fluorouracil. The preferred dose of 5-fluorouracil is 800-1200 mg/m² per day for 5 consecutive days.

Adenoviral therapy using the instant invention adenoviruses may be combined with other antineoplastic protocols, such as gene therapy. See, U.S. Pat. No. 5,648,478. As mentioned above, adenovirus constructs for use in the instant invention will exhibit specific cancer cell killing. Such constructs may also have, as mentioned above, prodrug activator genes, including thymidine kinase, cytosine deaminase, or others, that in the presence of the appropriate prodrug will enchance the antineoplastic effect of the invention adenovirus vectors. See, U.S. Pat. No. 5,631,236.

Also, in the event that the instant invention adenoviral mutants elicit an immune response that dampens their effect in a host animal, they can be administered with an appropriate immunosuppressive drug to maximize their effect. Alternately, a variety of methods exist whereby the exterior protein coat of adenovirus can be modified to produce less immunogenic virus. See, PCT/US98/0503 where it is shown that a major immunogenic component of adenovirus' exterior coat, hexon protein, can be genetically engineered to be less immunogenic. This is done by creating a chimeric hexon protein by substituting for normal viral hexon protein epitopes a sequence of amino acids not normally found in hexon protein. Such adenoviral constructs are less immunogenic than the wild type virus.

Another aspect of the instant invention is the incorporation of heterologous genes into, preferably, the E1 and/or E3 regions of the virus. Examples of such heterologous genes, or fragments thereof that encode biologically active peptides, include those that encode immunomodulatory proteins, and, as mentioned above, prodrug activators (i.e. cytosine deaminase, thymidine kinase, U.S. Pat. Nos. 5,358,866, and 5,677,178). Examples of the former would include interleukin 2, U.S. Pat. Nos. 4,738,927 or 5,641,665; interleukin 7, U.S. Pat. Nos. 4,965,195 or 5,328,988; and interleukin 12, U.S. Pat. No. 5,457,038; tumor necrosis factor alpha, U.S. Pat. Nos. 4,677,063 or 5,773,582; interferon gamma, U.S. Pat. Nos. 4,727,138 or 4,762,791; or GM-CSF, U.S. Pat. Nos. 5,393,870 or 5,391,485. Additional immunomodulatory proteins further include macrophage inflammatory proteins, including MIP-3. Monocyte chemotatic protein (MCP-3 alpha) may also be used. A preferred embodiment of a heterologous gene is a chimeric gene consisting of a gene that encodes a protein that traverses cell membranes, for example, VP22 or TAT, fused to a gene that encodes a protein that is preferably toxic to cancer but not normal cells.

To increase the efficacy of the invention adenoviral E1A mutant constructs they may be modified to exhibit enhanced tropism for particular tumor cell types. For example, as shown in PCT/US98/04964 a protein on the exterior coat of adenovirus may be modified to display a chemical agent, preferably a polypeptide, that binds to a receptor present on tumor cells to a greater degree than normal cells. Also see, U.S. Pat. No. 5,770,442 and U.S. Pat. No. 5,712,136. The polypeptide can be antibody, and preferably is single chain antibody.

A human patient or nonhuman mammal having a bronchogenic carcinoma, nasopharyngeal carcinoma, laryngeal carcinoma, small cell and non-small cell lung carcinoma, lung adenocarcinoma, hepatocarcinoma, pancreatic carcinoma, bladder carcinoma, colon carcinoma, breast carcinoma, cervical carcinoma, ovarian carcinoma, or lymphocytic leukemias may be treated by administering an effective antineoplastic dosage of an appropriate adenovirus. Suspensions of infectious adenovirus particles may be applied to neoplastic tissue by various routes, including intravenous, intraperitoneal, intramuscular, subdermal, and topical. An adenovirus suspension containing about 10³ to 10¹² or more virion particles per ml may be inhaled as a mist (e.g., for pulmonary delivery to treat bronchogenic carcinoma, small-cell lung carcinoma, non-small cell lung carcinoma, lung adenocarcinoma, or laryngeal cancer) or swabbed directly on a tumor site for treating a tumor (e.g., bronchogenic carcinoma, nasopharyngeal carcinoma, laryngeal carcinoma, cervical carcinoma) or may be administered by infusion (e.g., into the peritoneal cavity for treating ovarian cancer, into the portal vein for treating hepatocarcinoma or liver metastases from other non-hepatic primary tumors) or other suitable route, including direct injection into a tumor mass (e.g., a breast tumor), enema (e.g., colon cancer), or catheter (e.g., bladder cancer).

The invention now being fully described, it will be apparent to one of ordinary skill in the art that many changes and modifications can be made thereto without departing from the spirit or scope of the appended claims.

We claim:

1. An adenovirus comprising at least one mutation in the i-leader sequence of the viral major late transcriptional unit.

2. An adenovirus as described in claim 1, wherein said virus is Ad 5.

3. An adenovirus as described in claim 2, wherein said mutation is a C to T mutation at nucleotide 8350.

4. An adenovirus as described in claim 3, wherein said adenovirus is selected from the group consisting of Onyx 201 or Onyx 203.

5. An adenovirus as described in claim 2, further comprising a mutation that facilitates the replication of said adenovirus in cancer cells that functional lack a tumor suppressor protein.

6. An adenovirus as described in claim 5, wherein said tumor suppressor proteins comprise p53 and/or pRb.

7. An adenovirus as described in claim 6, wherein said mutation that facilitates the replication of said adenovirus in cancer cells that functional lack tumor suppressor proteins p53 and/or pRb comprise a mutation in the E1B and/or E1A regions of the adenovirus, respectively.

8. An adenovirus as described in claim 2, further comprising a heterologus gene.

9. An adenovirus as described in claim 8, wherein said heterologous gene encodes a negative selection gene or a cytokine.

10. An adenovirus as described in claim 2, further comprising a tissue specific promoter, or an E2F responsive element operably linked to an adenoviral gene that is essential for the replication of said adenovirus.

11. A method of killing cancer cells, comprising the steps of contacting said cancer cells with an adenovirus comprising at least one mutation in the i-leader sequence of the viral major late transcriptional unit for a time sufficient to kill said cancer cells.

12. A method of killing cancer cells as described in claim 11, wherein said mutation is a C to T mutation at nucleotide 8350.

13. A method of killing cancer cells as described in claim 12, wherein said adenovirus is selected from the group consisting of Onyx 201 or Onyx 203.
